(19) United States
(12) Patent Application Publication
(10) Pub. No.: US 2006/0153887 A1
(43) Pub. Date: Jul. 13, 2006

(54) Cosmetic article and process for obtaining a cosmetic article in a unit dose

(75) Inventor: Pablo Eugenio Ripoli, Santana do Parnaiba - SP (BR)

Correspondence Address: DARBY & DARBY P.C., P. O. BOX 5257, NEW YORK, NY, 10150-5257, US

(73) Assignee: MFR PRODUTOS PROMOCIONAIS LTDA, Santana do Parnaiba - SP (BR)

(21) Appl. No.: 11/183,233

(22) Filed: Jul. 14, 2005

(30) Foreign Application Priority Data
Jul. 16, 2004 (BR) PI 0404972-1

(51) Int. Cl. A61K 8/89; A61K 8/02

(52) U.S. Cl. 424/401; 424/70.12

(57) ABSTRACT

A COSMETIC ARTICLE AND PROCESS FOR OBTAINING A COSMETIC ARTICLE IN A UNIT DOSE, said cosmetic article comprising: a base substrate (10) having an upper face (11); a cosmetic product (20) which is deposited and retained, in a unit dose, onto a load region (13) of the upper face (11) of the base substrate (10); an oleophobic overlay film (30) covering the cosmetic product (20) and affixed to the upper face (11) of the base substrate (10) so as to be irreversibly removed from the latter upon access to the cosmetic product by the user; and an adhesive (40) applied and permanently adhered to the upper face (11) of the base substrate (10), in order to affix the overlay film (30) to the upper face (11) until the irreversible removal of said overlay film (30).

### FIELD OF THE INVENTION

The present invention refers, in general, to a cosmetic article and to a process for obtaining a cosmetic article in a small unit dose and particularly to a cosmetic article of the type comprising a base substrate, usually pasteboard or paper, onto which is applied and retained a unit individual dose of a cosmetic product in powder, eye shadow, blush, etc., which is covered by a transparent film overlay affixed to the base substrate.

### PRIOR ART

It is usual in the cosmetics industry the presentation of products in the form of powdered samples in disposable packages and in which small samples of cosmetic products are affixed to a substrate, usually through an adhesive, and covered by a transparent film.

The known solutions present disadvantages such as allowing the imperceptible violation of the packaged cosmetic product, since the overlay film is removably glued or otherwise sealed to the base substrate, and may be manually detached from the base substrate, providing access to the cosmetic product, and resealed to the base substrate, without notice of the violation of the packaged cosmetic product. These known solutions are not hygienic and safe to stop transmission of dermatosis and other diseases by the common and unaware use of a same dose of the cosmetic product by different users.

Besides the problem mentioned above, the solutions for packaging unit or reduced doses of powdered cosmetic products use adhesives or adhesive compounds to affix the cosmetic product to the substrate, which leads to further disadvantages such as the drying and hardening of the product, with the loss of softness, making difficult its use and application and allowing the occurrence of allergies in the users due to the use of adhesive for affixing said cosmetic products to the base substrate.

### OBJECTS OF THE INVENTION

It is therefore an object of the present invention the provision of a cosmetic article in a unit dose of the type considered herein and which overlay film is irreversibly removed from the base substrate upon the access to the cosmetic product by the user, making evident the opening of the package and avoiding its unnoticed violation.

It is a further object of the present invention to provide an article such as mentioned above, which avoids the use of adhesives for retaining the cosmetic product to the base substrate onto which the unit dose is deposited and consequently the drying of the cosmetic product and the possibility of provoking allergies on the user.

### SUMMARY OF THE INVENTION

These and other objects and advantages of the present invention are achieved by the provision of a cosmetic article in a unit dose, comprising: a base substrate having an upper face; a cosmetic product which is deposited and retained, in a unit dose, onto a load region of the upper face of the substrate; an oleophobic overlay film covering the cosmetic product and affixed to the upper face of the base substrate so as to be irreversibly removed from the latter upon access to the cosmetic product by the user; and an adhesive which is applied and permanently adhered to the upper face of the base substrate along a soldering region externally contouring the load region, in order to affix the overlay film to the upper face until the irreversible removal of said overlay film.

The present invention further provides a process for obtaining a cosmetic article in a unit dose, comprising the steps of: a—providing a base substrate in pasteboard, paper or the like having an upper face; b—applying an adhesive to the upper face of the base substrate along a soldering region externally contouring a load region defined on the upper face of the base substrate; c—allowing the adhesive to dry while it partially and superficially penetrates the upper face of the base substrate; d—depositing and retaining a unit dose of the powdered cosmetic product onto a load region of the upper face of the base substrate; e—applying an oleophobic overlay film on the upper face of the base substrate in order to cover the cosmetic product and to contact the adhesive previously applied and adhered to the base substrate; and f—providing the adhesion of the adhesive to the material of the overlay film so that the latter is irreversibly removed from the base substrate upon access to the cosmetic product by the user.

Other features and advantages of the present invention will become apparent from the detailed description of the invention and drawings illustrated below.

### BRIEF DESCRIPTION OF THE DRAWINGS

The invention will now be described in relation to the attached drawings, given by way of example of a possible construction for the subject cosmetic article and in which:

FIG. 1 is a perspective view of a plurality of cosmetic articles produced according to the present invention and having its respective base substrates united to each other in a single coupon by means of breakable weakened lines, said view further illustrating the basic film exploded in relation to the single coupon.

FIGS. 2 and 3 are perspective views of the coupon of cosmetic articles of FIG. 1, said views illustrating a sequence of individual removal of a cosmetic article from said coupon; and

FIG. 4 is an enlarged cross-sectional view of the coupon of cosmetic articles, taken along line IV-IV of FIG. 1, further illustrating the overlay film deposited and affixed on a base substrate represented by a coupon.

### DETAILED DESCRIPTION OF THE INVENTION

The present invention refers to a cosmetic article in a unit dose and formed by a reduced load of powder, in order to define an eye shadow, blush, etc.

According to the invention, the cosmetic article comprises: a base substrate 10 having an upper face 11 and a lower face 12; a cosmetic product 20 which is deposited and retained, in a unit dose, on the upper face 11 of the base substrate 10, in a load region 13 defined on said upper face 11; an overlay film 30, covering the cosmetic product 20 and having an oleophobic lower face 31, said overlay film 30 being affixed to the upper face 11 of the base substrate 10 to be irreversibly removed from the latter, upon access to the cosmetic product by the user; and an adhesive 40, which is applied and adhered to the upper face 11 of the base substrate 10 along a soldering region 14 externally contouring the load region 13, in order to affix the overlay film 30 to the upper face 11 of the base substrate 10 until the irreversible removal of the overlay film 30 with the correspondent removal of a superficial layer of the upper face 11 of the base substrate 10.

In the present invention, the base substrate 10 is made of a sheet of paper, pasteboard, couché paper or the like, with a thickness that may vary according to the use characteristics of the cosmetic article being formed, usually from 150 to 300 grams per square meter.

The base substrate 10 has its upper face 11 usually provided with a decorative printing including one or more colors and carried out by any known printing technique, such as offset, lithography, flexography, etc., with the care of defining the load region 13, onto which will be deposited the dose of cosmetic product 20 and presenting a previously defined contour.

The powdered cosmetic product 20 should be prepared to remain adequately aggregated when deposited on the load region 13 of the upper face 11 of the base substrate 10, maintaining the integrity of the dose upon the opening of the article by the removal of the overlay film 30.

The cosmetic product 20 has its load of powder humidified by a mixture of volatile silicone, cosmetic base and mineral oil, defining a pasty slurry. This controlled humidification allows the cosmetic dose to remain seated and adhered to the base substrate 10 with no need of applying adhesive between the cosmetic product 20 and the base substrate 10. The inexistence of adhesive as a retaining means of the cosmetic product 20 allows the unit dose to present characteristics of softness and application smoothness which are not found in cosmetic products affixed to the carrying substrate by means of adhesive application.

The cosmetic product 20 may be applied to the base substrate 10 by any known technique such as serigraphy, flexography or lithography.

The overlay film 30 is basically a barrier film, which is usually transparent, for the prompt visualization of the portion of cosmetic product 20 and obtained in oleophobic material, preferably PVC or polyester, to cover the cosmetic product 20 and keep it insulated from the environment.

The fixation of the overlay film 30 over the base substrate 10 is carried out along at least one soldering region 14, which is exterior and adjacent to the contour of the load region 13 receiving the cosmetic product 20 to be deposited, during the process of obtaining the subject cosmetic article, on the base substrate 10, in order to maintain the integrity of the contour of said load region 13.

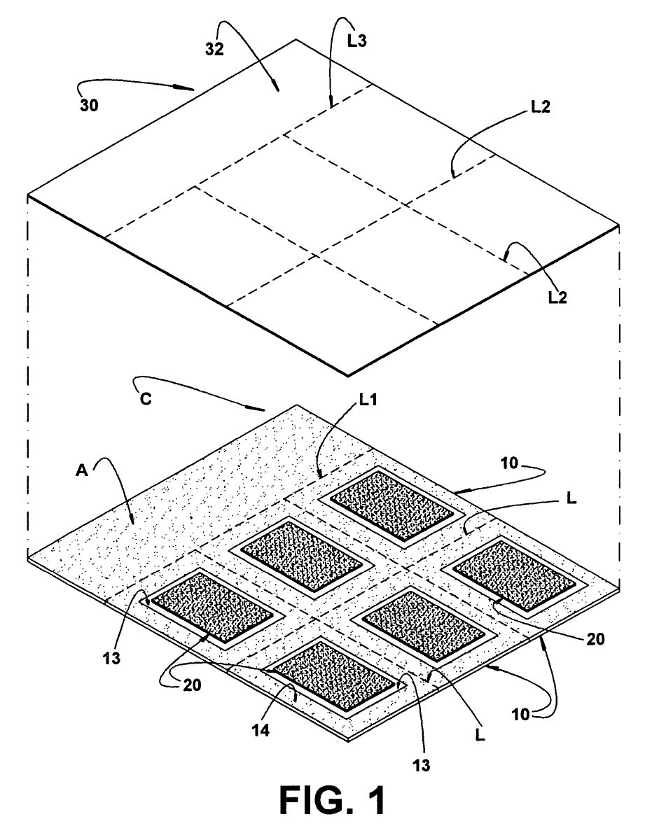

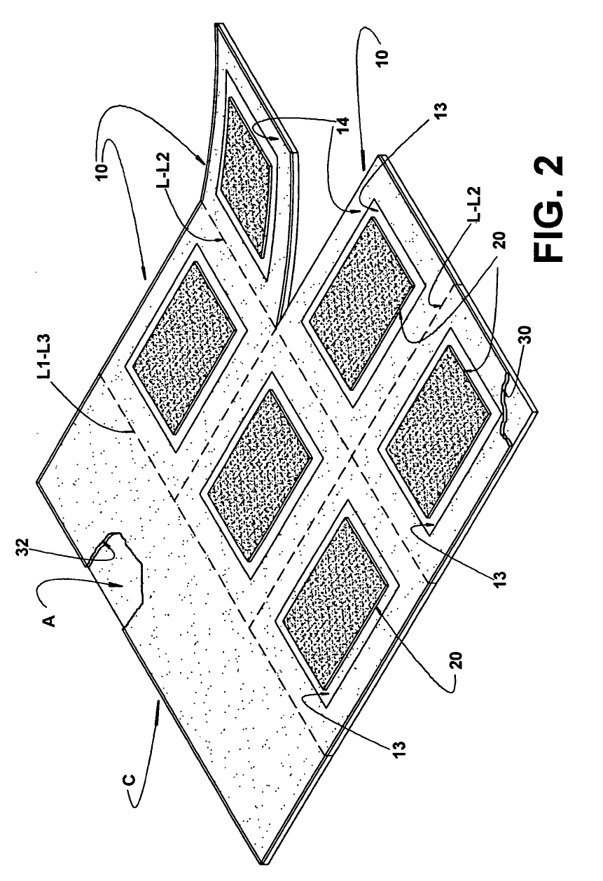

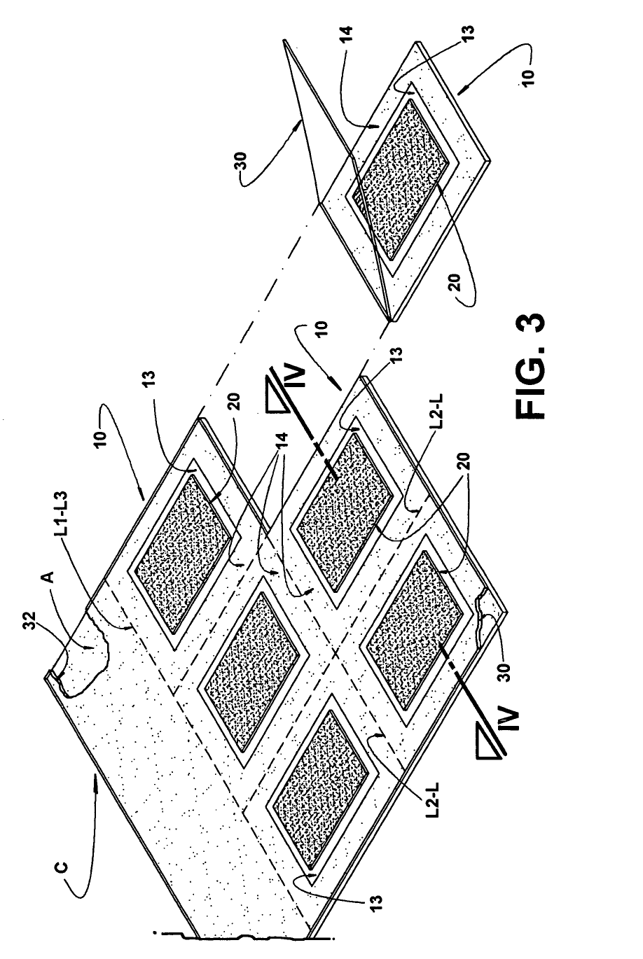

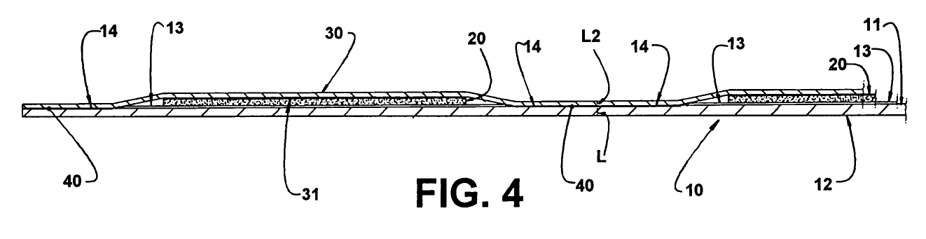

As illustrated in FIGS. 1-4, the fixation between the base substrate 10 and the overlay film 30 may be additionally provided by another soldering region 14 extending along the peripheral region of the base substrate 10. Other configurations of the soldering region 14 are possible to suit the particularities of the different applications of the cosmetic article.

An adhesive 40, preferably defined by a PVC based liquid varnish is applied along the soldering regions 14 defined on the upper face 11 of the base substrate 10. After being applied, the adhesive 40 is allowed to dry while penetrates partially and superficially in the upper face 11 of the base substrate 10. After the adhesive 40 has dried, the deposition of the unit dose of the cosmetic product 20 is provided on the respective load region 13 of the base substrate 10.

After the unit dose of the cosmetic product 20 has been deposited, the overlay film 30 is seated on the base substrate 10 and the soldering of the latter to the overlay film 30 is provided by thermo fusion or by applying high frequency to the adhesive 40 previously applied to the base substrate 10.

The activation of the adhesive causes the varnish in its composition to be soldered to the PVC of the overlay film 30, providing a solid union between the latter and the upper face 11 of the base substrate 10. Due to the soldering obtained between a superficial layer of the upper face 11 of the base substrate 10 and the overlay film 30 through the use of the cured adhesive 40, after the deposition of the cosmetic product 20, when the overlay film 30 is removed to provide access to the cosmetic product 20, the superficial layer of said upper face 11 of the base substrate 10 is removed together with the overlay film 30, making evident the opening of the cosmetic article and preventing the overlay film 30 from re-adhering to the base substrate 10.

As illustrated in the drawings, the subject cosmetic article may be marketed as coupons C of the same material defined to be used for making the base substrates 10, each coupon C being defined by one or more lines and columns of base substrates 10 united to each other through weakened lines L to be torn when the user desires to detach a cosmetic article from the coupon C.

The coupon C may further incorporate, through a respective weakened line L1, a tab A projecting outwardly from the contour defined by the plurality of cosmetic articles united to each other, and which is usually utilized for printing decorative motives and/or indicative of the features of the cosmetic product being offered. Normally, the tab A has its upper portion covered by a respective portion 32 of the overlay film 30, and being detachably united to the adjacent overlay films 30 by means of a weakened line L3.

In the case of providing the cosmetic articles in coupons C, each base substrate 10, separated from the others and from the tab A by respective weakened lines L, L1, carries on its upper face 11, a respective overlay film 30 which is separated from the overlay films 30 of the remaining cosmetic articles of the coupon C by means of respective weakened lines L2 which are broken upon the separation of one cosmetic article from the others.

While only one constructive form for the present cosmetic article has been illustrated herein, it should be understood that modifications in shape and relative arrangement of the different components may be carried out without departing the constructive scope defined in the following claims.

1. A cosmetic article in a unit dose, characterized in that it comprises: a base substrate (10) having an upper face (11); a cosmetic product (20) which is deposited and retained, in a unit dose, onto a load region (13) of the upper face (11) of the base substrate (10); an oleophobic overlay film (30) covering the cosmetic product (20) and affixed to the upper face (11) of the base substrate (10) so as to be irreversibly removed from the latter upon access to the cosmetic product by the user; and an adhesive (40) which is applied and permanently adhered to the upper face (11) of the base substrate (10) along a soldering region (14) externally contouring the load region (13), in order to affix the overlay film (30) to the upper face (11) until the irreversible removal of said overlay film (30).

2. Cosmetic article, according to claim 1, characterized in that the cosmetic article (20) is self fixable to the load region (13) of the upper face (11) of the base substrate (10), due to the humidification of the powder with a mixture of volatile silicone and mineral oil.

3. Cosmetic article, according to claim 1, characterized in that the cosmetic product (20) is a pasty slurry comprising powders, volatile silicones and mineral oils.

4. Cosmetic article, according to claim 1, characterized in that the cosmetic product (20) is deposited by known techniques such as serigraphy, flexography or lithography and retained, according to a predetermined contour, on the load region (13) of the upper face (11) of the base substrate (10).

5. Cosmetic article, according to claim 1, characterized in that the overlay film (30) is made of a transparent material.

6. Cosmetic article, according to claim 5, characterized in that the overlay film (30) is obtained from any of the materials defined by PVC and polyester, polypropylene, acetate.

7. Cosmetic article, according to claim 1, characterized in that the adhesive (40) is applied to the upper face (11) of the base substrate (10), in order to remove a superficial layer of said upper face (11) upon the irreversible removal of the overlay film (30) from the base substrate (10).

8. Cosmetic article, according to claim 7, characterized in that the adhesive (40) is adhered to the overlay film (30) by thermo fusion or by high frequency.

9. Cosmetic article, according to claim 1, characterized in that base substrate (10) is selected from one of the materials defined by paper, pasteboard, couché paper.

10. Cosmetic article, according to claim 1, characterized in that it comprises a plurality of base substrates (10) and respective overlay films (30) united to each other by means of weakened lines (L, L2) of a single coupon (C), said weakened lines being manually breakable by the user to detach each cosmetic article from the others.

11. Cosmetic article, according to claim 10, characterized in that the coupon (C) comprises a tab (A) projecting outwardly from the contour of the cosmetic articles and bonded thereto by means of a weakened line (L1, L3), also manually breakable.

12. A process for obtaining a cosmetic article in a unit dose, characterized in that it comprises the steps of: a—providing a base substrate (10) in pasteboard, paper or the like having an upper face (11); b—applying an adhesive (40) to the upper face (11) of the base substrate (10) along a soldering region (14) externally contouring a load region (13) defined on the upper face (11) of the base substrate (10); c—allowing the adhesive (40) to dry while it partially and superficially penetrates the upper face (11) of the base substrate (10); d—depositing using known techniques such as serigraphy, flexography or lithography and retaining a unit dose of the powdered cosmetic product (20) onto a load region (13) of the upper face (11) of the base substrate (10); e—applying an oleophobic overlay film (30) on the upper face (11) of the base substrate (10) in order to cover the cosmetic product (20) and to contact the adhesive (40) previously applied and adhered to the base substrate (10); and f—providing the adhesion of the adhesive (40) to the material of the overlay film (30) so that the latter is irreversibly removed from the base substrate (10) upon access to the cosmetic product (20) by the user.
